(19) United States
(12) Patent Application Publication
(10) Pub. No.: US 2004/0126772 A1
(43) Pub. Date: Jul. 1, 2004

(54) Method for maldi-tof-ms analysis and/or sequencing of oligonucleotides

(76) Inventors: Yoshihide Hayashizaki, Tsukuba-shi (JP); Tetsuyoshi Ono, Hitachinaka-shi (JP)

Correspondence Address: BIRCH STEWART KOLASCH & BIRCH, PO BOX 747, FALLS CHURCH, VA, 22040-0747, US

(21) Appl. No.: 10/433,899

(22) PCT Filed: Dec. 6, 2001
(86) PCT No.: PCT/JP01/10689

(51) Int. Cl. C12Q 1/68

(52) U.S. Cl. 435/6

(57) ABSTRACT

The present invention is related to a method for MALDI-TOF-MS analysis and/or sequencing of oligoribonucleotides. The present invention is also related to a method for determining the DNA nucleotide sequence using MALDI-TOF-MS, and a method for determining polymorphism using MALDI-TOF-MS. The present invention provides a kit for analyzing and/or sequencing a DNA template or a RNA transcription product and/or for determining polymorphism by MALDI-TOF-MS.

### FIELD OF THE INVENTION

The present invention relates to an improved method for the analysis and/or sequencing of oligonucleotides by using Matrix-assisted laser desorption/ionization time-of-flight mass spectrometry (MALDI-TOF-MS) methodology. In particular, the invention relates to modified ribonucleotides, which reduce or eliminate the signal intensity drop-off during the MALDI analysis and/or sequencing.

### BACKGROUND ART

The Human Genome Project is in the final stages of sequencing the human genome. One of the post-human genome projects would be to re-sequence a specific site for comparing each different person or species as well as for the determination of Single Nucleotide Polymorphisms (SNPs). Such a re-sequencing system must be very speedy and cost effective.

MALDI-TOF-MS (Smith, L. M., Science, 1993, 262, 530; and Hillenkamp et al., Biological Mass Spectrometry, Burlingame and McCloskey Editors, Elsevier Science Publishers, Amsterdam, 1990, pp.49-60), has evolved into a rapid, accurate, and sensitive method for the mass analysis of high molecular weight synthetic and biologically important polymers. MALDI-TOF-MS represents an advantageous methodology for analyzing and sequencing oligonucleotides as well as for the determination of SNPs.

MALDI-TOF-MS has the advantage of enabling very fast DNA sequencing and not requiring gels or fluorescent-dyes.

Short DNA re-sequencing systems based on the Sanger method have been performed with MALDI-TOF-MS (Monforte, J. A., and Becker, C. H., Nature Med., 1997, 3, 360-362; Kirpekar, F. et al., Nucleic Acids Res., 1998, 26, 2554-2559). The longer the length of DNA that can be sequenced using MALDI-TOF-MS analysis, the greater the advantages MALDI-TOF-MS will gain as an alternative technique in the area of DNA sequencing.

However, substantial limitations in the approach of analyzing DNA by MALDI-TOF-MS have been revealed. One of these limitations is that the DNA molecules fragment substantially during the course of the MALDI process. The DNA exhibits not only the intact parent ion signal but also some other ion signals caused from the DNA fragmentation, as base loss and backbone cleavage. A model was developed for the fragmentation mechanism (Zhu, L.; et al., J. Am. Chem. Soc. 1995, 117, 6048-6056) in which the initiating step of DNA fragmentation during MALDI is protonation of the nucleobase moiety, which weakens the N-glycosidic linkage causing base loss with concomitant formation of a carbocation at the 1′ position of the deoxyribose moiety. A subsequent rearrangement leads to backbone cleavage at the 3′ carbon-oxygen bond.

The stabilization of DNA during the MALDI analysis has been thought to have utility for DNA sequencing or other nucleic acid analyses.

U.S. Pat. No. 5,691,141 describes a method for sequencing DNA, based on the Sanger methodology. This method involves introducing mass modifications into the oligonucleotide primer, the chain-terminating nucleoside triphosphates and/or the chain-elongating nucleoside triphosphates, or by using mass-differentiated tag probes hybridizable to specific tag sequences. As nucleotide modifications, U.S. Pat. No. 5,691,141 describes primers modified by glycine residues at the 5′-position of the sugar moiety of the terminal nucleoside; primers at C-5 of the heterocyclic base of a pyrimidine nucleoside with glycine residues, with β-alanine residues, with ethylene glycol monomethyl ether, with diethylene glycol monomethyl ether; primers mass-modified at C-8 of the heterocyclic base of deoxyadenosine with glycine or glycylglycine; primers mass-modified at the C-2′ of the sugar moiety of 2′-amino-2′-deoxythymidine with ethylene glycol monomethyl ether residues; DNA primers mass-modified in the internucleotidic linkage via alkylation of phosphorothioate groups (according to the procedure described in Slim G. and Gait M. J., Nucleic Acids Research, 1991, vol.19, No.6, 1183-1188); 2′-amino-2′-deoxyuridine-5′-triphosphate and 3′-amino-2′,3′-dideoxythymidine-5′-triphosphate mass-modified at the 2′-amino or 3′-amino function with glycine or β-alanine residues; deoxyuridine-5′-triphosphate mass-modified at C-5 of the heterocyclic base with glycine, glycyl-glycine and β-alanine residues; 8-glycyl-2′-deoxyadenosine-5′-triphosphate and 8-glycyl-glycyl-2′-deoxyadenosine-5′-triphosphate; and chain-elongating 2′-deoxy-thymidine-5′-(alpha-S—)triphosphate and chain-terminating 2′,3′-dideoxythymidine-5′-(alpha-S—)triphosphate and subsequent alkylation with 2-iodoethanol and 3-iodopropanol.

However, DNA sequencing by mass spectrometry using the modified oligonucleotides described U.S. Pat. No. 5,691,141 has the problems of signal intensity drop-off and base loss. This disadvantage does not allow sequencing in an efficient way, especially for the sequencing of longer DNA sequences (Taranenko, et al., Nucleic Acids Res., 1998, 26, 2488-2490).

Schuette J. M. et al., (J. Pharm. Biomed. Anal. 1995, 13, 1195-1203) suggest the use of MALDI-TOF-MS, previously used to sequencing natural phosphodiester DNA sequences, for sequencing DNA comprising alpha-phosphorothioate deoxyribonucleotides (S-dNTPs). However, the data presented in this document (for example FIG. 2 of Schuette at al.) show that the use of S-dNTPs is not efficient for sequencing analysis by using MALDI.

The unsuitability of the incorporation of phosphorothioate nucleotides into DNA for MALDI sequencing analysis have also been confirmed by the present inventors. In the present application, FIGS. 1A and 1B (referring to oligo DNA comprising S-dNTPs and referred to hereafter as S-DNAs) compared to FIGS. 1C and 1D (referring to phosphodiester DNAs) show that the substitution of phosphodiester to phosphorothioate in DNA sequences increases fragmentation when spectra of the 20 mer and 30 mer were focused. Then, the signal intensity drop-off with increasing mass range as shown by oligo S-DNA was much more dramatic than that shown by DNA.

The above data confirm the necessity, in this field of investigation, of developing a new approach to MALDI-TOF-MS analysis methodology which will be able to remedy the signal intensity drop-off and allow the sequencing of much longer oligonucleotides sequences.

### SUMMARY OF THE INVENTION

The present inventors have surprisingly found that alpha-phosphorothioate ribonucleotides having a 2′-electronegative substituents referred to hereafter as S-2′-e-NTPs (preferably S-2′-fluoro-ribonucleotides (S-2′-F-NTPs), or S-2′-OH-ribonucleotides (S-NTPs)), and arabino-ribonucleotides (ara-NTPs) can be advantageously used in MALDI-TOF-MS analysis showing a resistance to signal intensity drop-off.

Accordingly, the present invention refers to a method for MALDI-TOF-MS analysis of RNA sequences, fragments or transcripts (in general oligoribonucleotides) which method utilizes at least one ribonucleotide selected from the group consisting of S-2′-e-ATP, -CTP, -GTP, -UTP and derivatives thereof (preferably S-2′-F-NTPs or S-NTPs).

The present invention also refer to a method for MALDI-TOF-MS analysis of RNA sequences, fragments or transcripts (in general oligoribonucleotides) which method utilizes at least one ribonucleotide selected from the group consisting of ara-ATP, -CTP, -GTP, -UTP and derivatives thereof.

Further, the present invention relates to a method for determining DNA nucleotide sequences using the MALDI-TOF-MS comprising:

a) providing ribonucleosides triphosphates or alpha-thio-substituted (chain-elongating ribonucleotides) selected from ara-NTPs or S-2′-e-NTPs (preferably S-2′-F-NTPs or S-NTPs) as above defined;

b) reacting said chain-elongating ribonucleotides with one or more kinds of 3′-dNTP derivatives (chain terminating ribonucleotides) in the presence of an RNA polymerase and a DNA template comprising a promoter sequence for the RNA polymerase to obtain an oligoribonucleotide transcription product; and

c) analyzing said oligoribonucleotide transcription product by MALDI-TOF-MS and determining the sequence of the transcription product and of the DNA template.

The invention also relates to a method for the determination of SNPs using MALDI-TOF-MS and S-2′-e-NTPs (preferably S-2′-F-NTPs or S-NTPs) or ara-NTPs.

The invention further refers to a kit for sequencing DNA templates or RNA transcription products by MALDI-TOF-MS, comprising:

i) a set of chain-elongating ribonucleotides modified according to the present invention (as above indicated at step a)) for synthesizing a RNA transcription product;

ii) one or more chain-terminating ribonucleotides for terminating the synthesis of the RNA transcription product and generating sets of base-specific terminated complementary ribonucleotide transcription fragments; and

iii) a RNA polymerase.

The kit above disclosed can also optionally further comprises (iv) one or more matrices for MALDI-TOF-MS analysis.

The invention also refers to a kit for the determination of SNPs using MALDI-TOF-MS comprising the elements (i)-(iii) and the optional (iv) as above disclosed.

### BRIEF DESCRIPTION OF THE DRAWINGS

FIGS. 1A and 1B: UV-MALDI mass spectra of equimolar mixtures of S-DNA obtained using 3-HPA (panel A) and THAP (panel B) as the matrix, respectively. The volume (μl) of sample/matrix on the probe tip (A) and (B) was: 0.5/1.0.

FIGS. 1C and 1D: UV-MALDI mass spectra of equimolar mixtures of DNA obtained using 3-HPA (panel C) and THAP (panel D) as the matrix, respectively. The volume (μl) of sample/matrix on the probe tip (C) and (D) was: 0.5/1.0.

FIGS. 2A and 2B: UV-MALDI mass spectra of equimolar mixtures of 2′-F-RNA obtained using 3-HPA (panel A) and THAP (panel B) as the matrix, respectively. The volume (μl) of sample/matrix on the probe tip (A) and (B) was: 0.8/0.8.

FIGS. 2C and 2D: UV-MALDI mass spectra of equimolar mixtures of RNA obtained using 3-HPA (panel C) and THAP (panel D) as the matrix, respectively The volume (μl) of sample/matrix on the probe tip (C) and (D) was: 0.8/0.8.

FIG. 3: Configuration of (A) deoxy-; (B) ribo-; (C) 2′-fluoro-; (D) arabino-: (E) phosphorothioated deoxy-; (F) phosphorothioated ribo-; (G) phosphorothioated 2′-fluoro-; and (H) phosphorothioated 2′-electronegative (2′-e) substituent oligonucleotide.

FIG. 4. UV-MALDI mass spectra of equimolar mixtures of S-RNA obtained using 3-HPA (panel A) and THAP (panel B) as the matrix, respectively. The volume (μl) of sample/matrix on the probe tip was (A): 0.5/1.0, (B): 0.8/0.8. The peaks indicated as [20 mer+2H]²⁺ and [30 mer+2H]²⁺ refer to a double positive charge effect.

FIG. 5. UV-MALDI mass spectra of equimolar mixtures of S-2′-F-RNA obtained using 3-HPA (panel A) and THAP (panel B) as the matrix, respectively. The volume (μl) of sample/matrix on the probe tip was (A): 0.5/1.0, (B): 0.8/0.8.

FIG. 6. UV-MALDI mass spectra of crude (not purified) S-2′F-RNA 10 mer (panel A), 20 mer (panel B), and 30 mer (panel C) obtained using 3-HPA as the matrix. The concentration (A): 250 μM, (B): 250 μM, (C): 250 μM was calculated as the concentration of the pure end product (A): 10 mer S-2′F-RNA, (B): 20 mer S-2′F-RNA, (C): 30 mer S-2′F-RNA, respectively. The volume (μl) of sample/matrix on the probe tip (A), (B), and (C) was: 0.5/1.0.

FIG. 7. UV-MALDI mass spectra of equimolar mixtures of (A) and (B): CH₃S-RNA-N⁺ obtained using 3-HPA without ammonium citrate dibasic (panel A) and 2,5-DHBA (panel B) as the matrix. The volume (μl) of sample/matrix on the probe tip was (A): 1.0/1.0, (B): 0.5/1.0.

FIG. 8. UV-MALDI mass spectra of equimolar mixtures of ara-RNA obtained using 3-HPA (panel A) and THAP (panel B) as the matrix, respectively. The volume (μl) of sample/matrix on the probe tip were (A): 0.5/2.0, (B): 0.5/1.5.

### DETAILED DESCRIPTION OF THE INVENTION

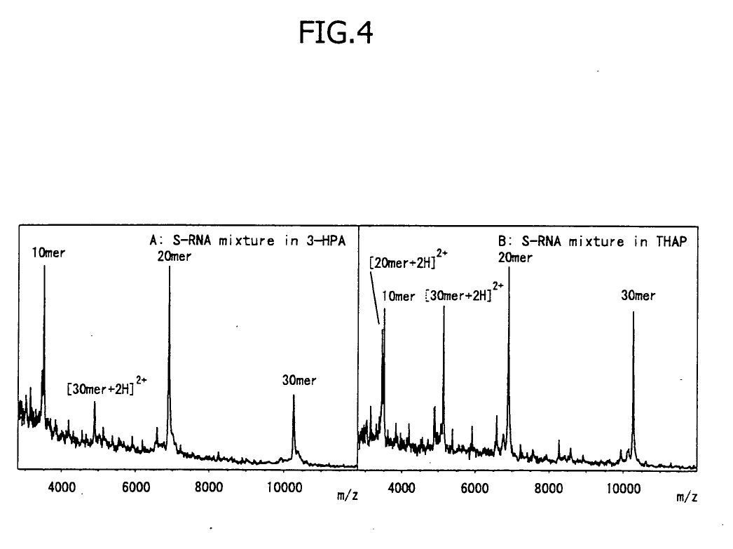

The present invention discloses a method for MALDI-TOF-MS analysis and/or sequencing of RNA sequences, fragments or transcripts (in general oligoribonucleotides) which method utilizes at least one ribonucleotide selected from S-2′-e-NTPs (preferably S-2′-F-NTPs or S-NTPs) and ara-NTPs. Examples of analysis of oligoribonucleotide ladders according to the present invention are reported in FIGS. 4, 5, 6 and 8.

The modified ribonucleotides useful in the MALDI-TOF-MS method according to the present invention, are indicated as S-2′-e-NTPs and ara-NTPs, while the oligo comprising the NTPs are indicated as oligo S-2′-e-RNA and oligo ara-RNA, respectively.

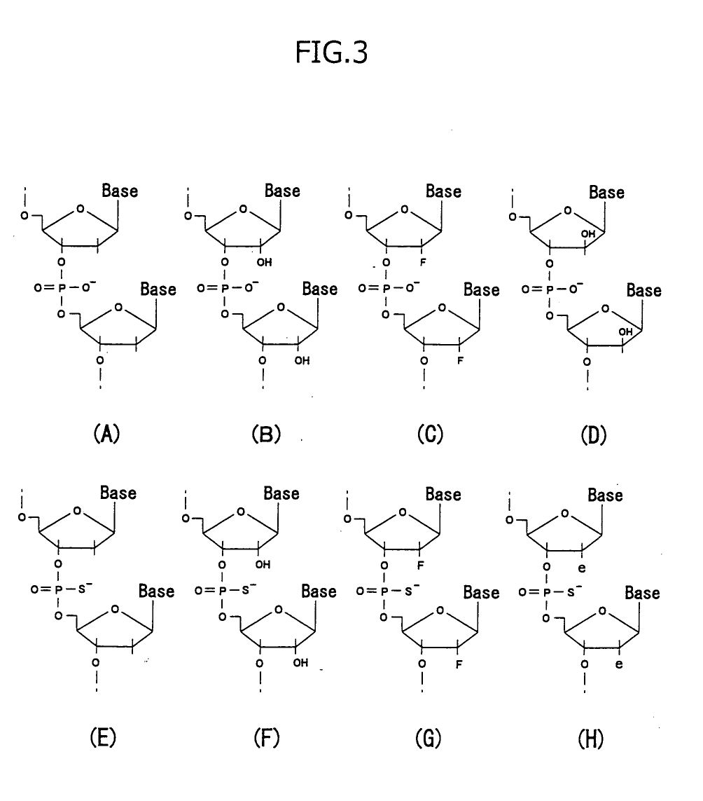

An oligo chemical formula comprising S-2′-e-NTPs is described in FIG. 3(H). The term “S” refers to the alpha-phosphothioate backbone, and the substituent “e” refers to a strong electronegative substituent at position 2′ of the ribose moiety. The electronegative substituent is preferably selected from the group consisting of F, Cl, NH₂, N₃ and OH (see Table III of Guschlbauer W. and Jankowski K., Nucleic Acid Research, 1980, volume 8, number 6, 1421-1433) but it is not limited to these preferred substituents. Other strong electronegative substituents can also be used. When “e” is —OH, the ribonucleotide is indicated as S-NTP (in this case the oligo will be indicated as oligo S-RNA).

The ribonucleotide S-2′-e-NTPs comprise different nitrogenous bases, that is adenine, guanine, cytosine, uracil and/or their derivatives. Accordingly, the compound S-2′-e-NTP can be also generally indicated as S-2′-e-ATP, S-2′-e-GTP, S-2′-e-CTP, S-2′-e-UTP.

Kawasaki A. M., et al., (J. Med. Chem., 1993, 36, 831-841) have disclosed S-2′-F-NTP and prepared oligo able to retain binding affinity to RNA targets. However, there has been no disclosure or suggestion of using this ribonucleotide or oligo for MALDI-TOF-MS analysis or sequencing. With reference to the S-2′-F-NTPs and S-2′-F-RNA according to the present invention, they have been prepared by TriLink BioTechnologies (San Diego, Calif.).

Oligo S-RNAs have been disclosed and synthesized by Slim G. and Gait M. J., (Nucleic Acids Research, 1991, vol.19, No.6, 1183-1188) in the study of the mechanism of cleavage of hammerhead ribozymes. However, there has been no disclosure or suggestion of use of this oligo in MALDI systems.

S-NTPs according to the present invention comprise different nitrogenous bases adenine, guanine, cytosine, uracil and/or their derivatives and are represented as S-ATP, S-CTP, S-GTP, S-UTP and derivatives thereof.

The present inventors have found that an oligo comprising at least one kind of S-NTP (formula F of FIG. 3) shows a resistance to signal intensity drop-off MALDI-TOF-MS analysis and/or sequencing (FIG. 4).

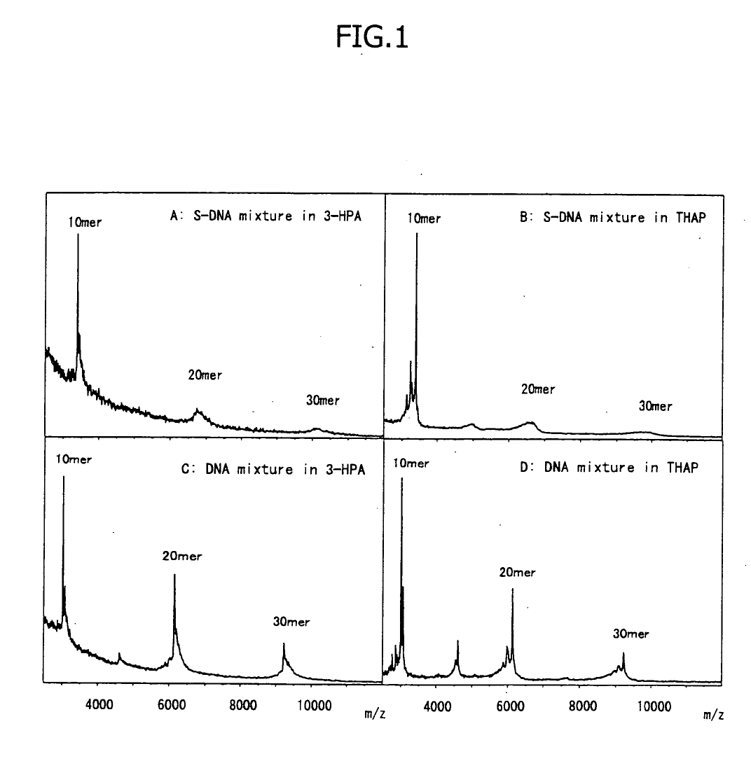

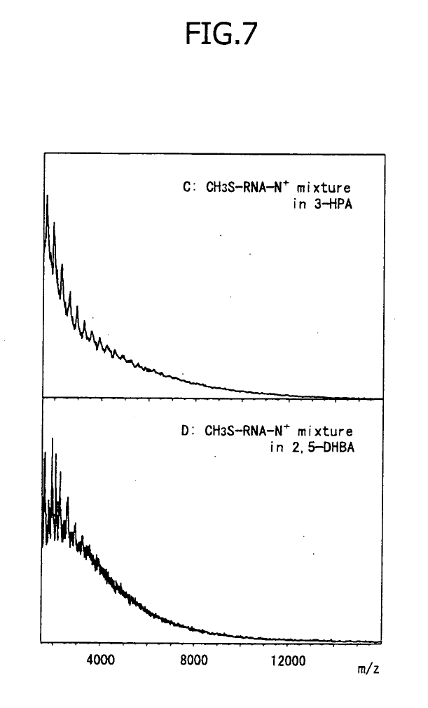

The advantageous use of S-RNA in analysis and sequencing with MALDI-TOF-MS was not predictable since the introduction of the alpha-phosphorothio group in DNA (see FIG. 3E and FIGS. 1A, 1B) as well as the introduction of an alkyl-thio- in the alpha phosphoric group of RNA (see FIG. 7) did not exhibit resistance to signal intensity drop-off in MALDI-TOF-MS analysis and/or sequencing.

With reference to the oligo S-2′-e-RNA, a preferred example is the oligo S-2′-F-RNA comprising at least one S-2′-F-NTP selected from the group consisting of S-2′-F-ATP, S-2′-F-CTP, S-2′-F-GTP, S-2′-F-UTP and derivatives thereof.

With reference to the fluoro substituent at position 2′ of the ribose, Tang Wei et al. (Anal. Chem., 1997, 69, 302-312) proposed that a deoxyribonucleotide (dNTP) having a fluorine moiety substituted at the 2′ carbon stabilizes the DNA sequence to fragmentation and thus extending the accessible mass range. However, a recent study (Scalf, M. Ph.D. thesis, University of Wisconsin, Madison, Wis., 2000) demonstrated that 2′-fluoro-electronegative substituents do not alleviate signal intensity drop-off. This may make the sequencing of DNA sequences disadvantageous in MALDI analysis.

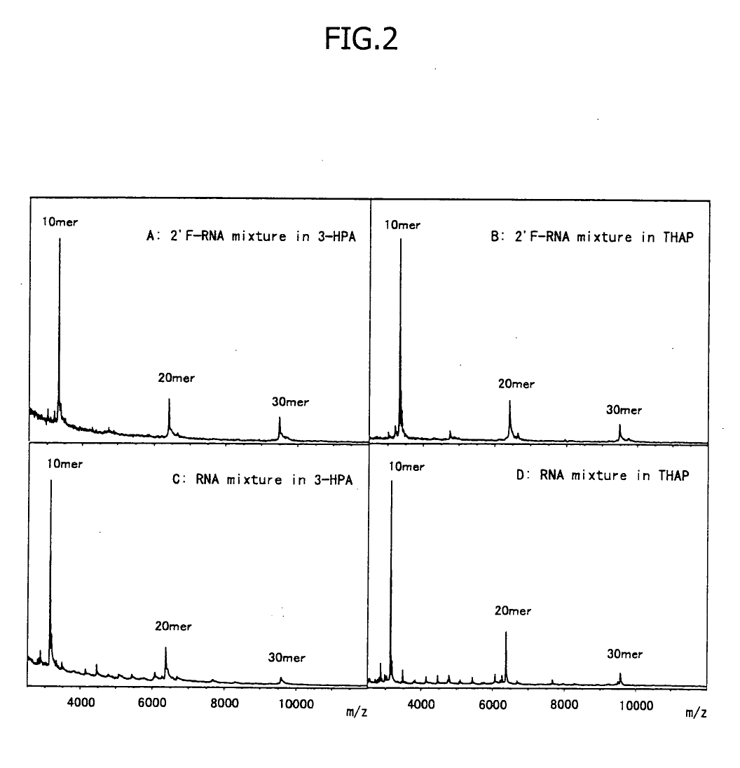

The present inventors have further investigated the effect of the introduction of 2′-fluoro ribonucleotides (2′-F-NTPs) into an oligo RNA (2-F′-RNA) and found that the result of MALDI sequencing method using oligo 2′-F-RNA (FIGS. 2A and 2B of the present application) does not differ from that of RNA sequences (FIGS. 2C and 2D) and does not alleviate signal intensity drop-off for long RNA sequences.

The 2′-F-RNAs used in the experimental part of the present application were prepared by TriLink BioTechnologies (San Diego, Calif.), synthesized in form of 2′-fluoro-(C)ₙT. The dTTP (which is not 2′-fluoro modified) was employed as starting nucleotide, binding the CPG support. On it the 2′-fluoro-CTPs were added according to the usual and well known technique in the art, described for example in “Current Protocols in Molecular Biology”, Vol.I, Section V, Unit 2.11, John Wiley & Sons, Inc. Oligomers of 2′-fluoro-(C)₁₀T, —(C)₂₀T and —(C)₃₀T were synthesized. These oligomers were referred to as 2′-F-RNA 10 mer, 20 mer and 30 mer, respectively, even if the real size of these oligomers is 11, 21 and 31 for the presence of T at the 3′ end. In fact, dTTP is not modified and the present invention relates to the 2′-electronegative- and ara-substituents.

S-2′-F—(C)ₙTs and ara(C)ₙTs were also synthesized in the same way as illustrated for 2′-F—(C)ₙTs. Also in these cases the oligomers will be referred to as 10 mer, 20 mer and 30 mer, even if the real size of them is 11, 21 and 31 because of the presence of T at the 3′ end.

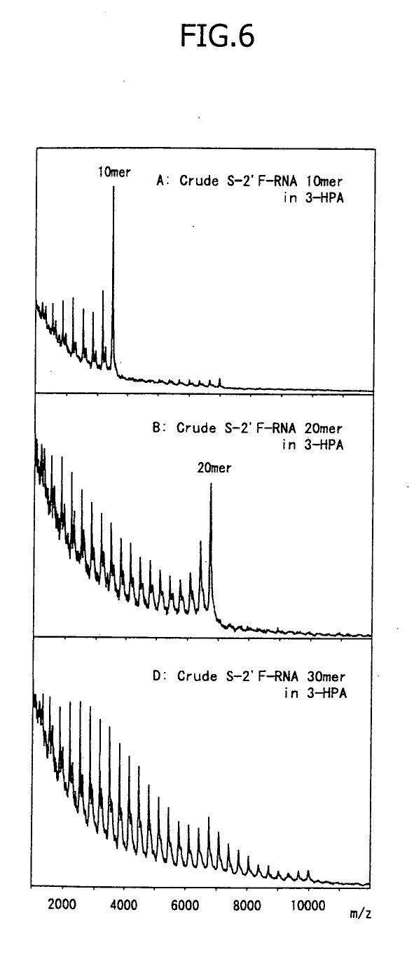

Similarly, the oligomer of FIG. 6, will be referred to 1-10 mer, 1-20 mer and 1-30 mer, even if their real size is 1-11, 1-21 and 1-31, because of the presence of T at the 3′, end.

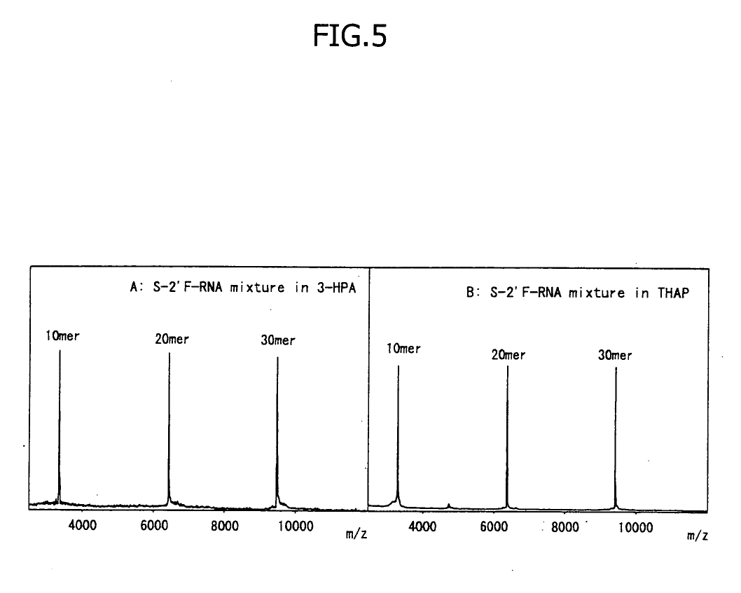

With reference to the method according to the present invention for analysis of oligos comprising S-2′-F-NTPs, FIGS. 5 and 6 clearly show that the introduction of the alpha-phosphorothio group in combination with the fluoro substituent at position 2′ of the ribose is particularly useful and efficient in the MALDI-TOF-MS analysis and/or sequencing showing a resistance to the signal intensity drop-off. This is a surprising effect since the introduction of an alkyl-S group into a ribonucleotide (see FIGS. 7C and D) as well as the introduction of a fluoro at 2′-position of a ribonucleotide (see FIGS. 2A and 2B) demonstrated a persistence of signal intensity drop-off.

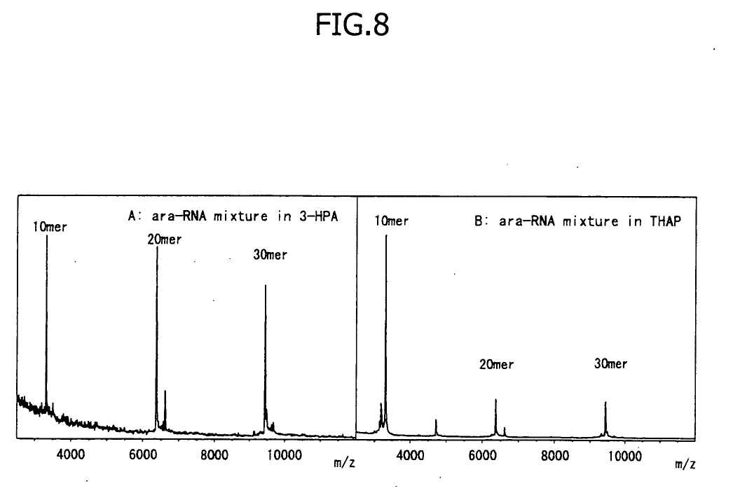

The present inventors also found that oligos comprising 2′-epimer-ribonucleotides (also known both as arabino-NTPs and ara-NTPs)(formula D of FIG. 3) show a resistance to signal intensity drop-off in MALDI-TOF-MS analysis and/or sequencing. These ribonucleotides comprise the different nitrogenous bases adenine, guanine, cytosine, uracil and/or their derivatives as described for compound S-2′-e-RNA. The resistance to signal intensity drop-off is shown in FIG. 8 for ORNs (oligoribonucleotides) ara-RNA.

Ara-RNA can be prepared, for example, according to Beardsley, G. P., et al., Nucleic Acids Res., 1988, 16, 9165-9176; Tang, W., et al., Anal. Chem., 1997, 69, 302-312. The ara-RNA was disclosed in Tang et al., but it was observed that the arabino-nucleosides showed base loss peaks as in FIGS. 3B, 4, 6B, and 8 (of the Tang et al. document). This indicated that the stabilization by this modification was less than complete (see page 311, left column, lines 16-20 of the Tang et al. document). Therefore, the ara-RNA was considered not useful for MALDI-TOF-MS analysis or sequencing. On the contrary, the present inventors have demonstrated that an ara-RNA ladder comprising ara-ribonucleotides, preferably in the presence of 3-HPA as MALDI matrix, show resistance to signal intensity drop-off using MALDI-TOF-MS analysis or sequencing.

The ribonucleotides described above are used to prepare oligoribonucleotide sequence products, which can be defined as S-2′-e-RNA (preferably S-2′-F-RNA or S-RNA) and ara-RNA. An oligoribonucleotide sequence product (ORN), according to the present invention, can be any ORN comprising the alpha-thio and/or arabino modified ribonucleotides according to the invention, such as, for example, an oligoribonucleotide ladder, an oligoribonucleotide fragment or complete sequence of a gene, a RNA transcript product of an Expressed Sequence Tag (EST) or a full-length RNA sequence, a fragment of a RNA transcript of t-RNA, r-RNA, m-RNA or a primer.

A RNA fragment such as a ladder can be prepared, for example, by providing at least one kind of the alpha-thio and/or arabino modified ribonucleotide according to the invention in order to form an ORN according to the standard technologies known in the art (for instance by chemical incorporation of modified ribonucleotides or by the Transcriptional Sequencing (TS) method below described). Accordingly, the present invention also refers to RNA sequences or fragments or transcript products (oligoribonucleotides) comprising ribonucleotides alpha-thio and/or arabino modified according to the invention, as above described.

Examples of analysis of oligoribonucleotide ladders are reported in FIGS. 4, 5, 6 and 8 showing a high resolution of the peaks indicating purity of the ladders analyzed. The analysis can also be used for determining the mass of the ladders.

The data of FIGS. 4, 5, 6 and 8 compared to the data of “control” FIGS. 1, 2 and 7 demonstrate that the introduction of alpha-thio phosphoric ribonucleotides into RNA ladders significantly reduced signal intensity drop-off (FIG. 4). It was an unexpected result in view of the fact that both the introduction of alpha-phosphorothioated dNTP into DNA (S-DNA) and the introduction of an alkyl-thio phosphoric NTP into RNA (CH₃S-RNA) ladders showed a high signal intensity drop-off (see FIGS. 1A, 1B and 7A, 7B, respectively).

The combination of both an alpha-thio phosphoric group and a strong electronegative substituent (preferably fluoro) at the 2′ position, introduced into a ribonucleotide renders a oligoribonucleotide (RNA) sequence comprising said ribonucleotides particularly useful for MALDI-TOF-MS analysis and/or sequencing. Accordingly, FIG. 5 shows that all the oligomers (10 mer, 20 mer and 30 mer) have a clear resistance to the signal drop-off compared to FIG. 4, wherein oligo 30 mer show in 3-HPA a signal drop-off effect. S-2′-F-RNA is particularly stable and resistant to base loss and exhibits lower background noise than S-RNA.

Comparison between FIG. 5 and FIGS. 2A,B clearly show an improved resistance to signal drop-off for 20 mer and 30 mer ladders of 2′-F-RNAs.

FIG. 6 is an UV-MALDI-TOF-MS mass spectra for fragments of 1-10 mer (panel A), fragments of 1-20 mer (panel B) and fragments 1-30 mer (panel C). The panels of FIG. 6 show that the resolution of the peaks was good and the method has proved to efficiently sequence the provided fragments.

FIG. 8 shows that ara-RNA 10 mer, 20 mer and 30 mer ladders, preferably in presence of matrix 3-HPA, have a resistance to signal drop-off and therefore ara-NTPs can be efficiently used in MALDI-TOF-MS analysis and/or sequencing.

The matrices employed in the MALDI-TOF-MS method according to the present invention can be selected from the matrices usually employed in MALDI methodology, for example 3-HPA, THAP, 2,5-DHBA (Zhu, Y. F., et al., Rapid Commun. Mass Spectrometry, 1996, 10, 383-388; Tang, W., et al., Anal. Chem., 1997, 69, 302-312). The selection of a specific matrix in particular experimental conditions could be important in MALDI methodology for obtaining a good resolution of the signal and a resistance to signal drop-off. For example, in MALDI analysis arabino-10 mer, 20 mer and 30 mer (FIG. 8A), the selection of the matrix 3-HPA is considered preferably advantageous.

With reference to the sequencing of RNA sequences or fragments, sequencing methodologies have been described in the art, for example by Faulstich, K., et al., Anal. Chem., 1997, 69, 4349-4353; and Wouner, K., et al., Nucleosides & Nucleotides, 1997, 16, 573-577. However, these methods refer to RNA genomic fragment sequencing and cannot be used for purpose of the present method, which requires the introduction of the modified ribonucleotides into oligoribonucleotides.

Accordingly, another aspect of the present invention relates to a method for determining DNA nucleotide sequence using the method called “transcriptional sequencing” (TS) and MALDI-TOF-MS.

The TS method is described in Sasaki, N. et al. (Proc. Natl. Acad. Sci. USA, 1998, 95, 3455-3460), and also in U.S. Pat. No. 6,074,824, and PCT application WO 99/02729. TS involves a method for determining the DNA nucleotide sequence of a DNA template, comprising I) providing ribonucleoside-5′-triphosphates (also known as chain-elongating ribonucleotides) selected from the group consisting of ATP, GTP, CTP, UTP and derivatives thereof; II) reacting said ribonucleotides with one or more 3′-dNTP derivatives (chain-terminating ribonucleotides) in presence of RNA polymerase and the DNA template fragment or sequence comprising a promoter sequence for the RNA polymerase; and III) separating the resulting RNA transcription products and determining the ribonucleotide sequence of the RNA transcript (and of the DNA template).

According to a further aspect, the present invention relates to a method comprising the steps of:

a) providing ribonucleotides, such as S-2′-e-NTPs (preferably S-2′-F-NTPs, S-2′-Cl-NTPs, S-2′-NH₂-NTPs, S-2′-N₃-NTPs or S-NTPs) or ara-NTPs;

b) reacting the ribonucleotides of step a) with one or more kinds of 3′-dNTP derivatives in the presence of RNA polymerase and a DNA template comprising a promoter sequence for the RNA polymerase to obtain an oligoribonucleotide transcription product;

c) analyzing said oligoribonucleotide transcription product by MALDI-TOF-MS and determining the transcription product sequence and DNA template sequence.

The oligoribonucleotide transcription product can be preferably purified before applying the MALDI step (Wu, Q. et al., Rapid Commun. Mass Spectrum., 1996, 10, 835-838).

Optionally, the DNA template can be subjected to an amplification step before performing TS, as disclosed in U.S. Pat. No. 6,074,824.

In the step a) of the above method, the S-2′-e-NTPs are selected from the group consisting of S-2′-e-ATP, S-2′-e-GTP, S-2′-e-CTP, S-2′-e-UTP, and derivatives thereof (wherein “e” is preferably F, Cl, NH₂, N₃ or OH); and the ara-NTPs are selected from the group consisting of ara-ATP, ara-GTP, ara-CTP, ara-UTP and derivatives thereof.

The phrase “derivative thereof” is intended to encompass NTPs or dNTPs comprising any modification known in the art, for example, those having the modified bases listed in Table 13-3 of patent in Version 2.1, User Manual, U.S. Patent and Trademark Office, or WIPO Standard ST.25 (1998), Appendix 2, Table 2.

The 3′-dNTP derivatives (also known as chain-terminating ribonucleotides) are selected from the group consisting of 3′-dATP, 3′-dGTP, 3′-dCTP, 3′-UTP and derivatives thereof, having the modification as above disclosed S-2′-e-, S- , and arabino. In simple words, they correspond to the modified ribonucleotides according to the present invention having a deoxy at 3′-position, so that they terminate the ribonucleotide transcript synthesis. They can also be indicated as S-2′-e-3′-dNTPs, S-3′-dNTPs, ara-3′-dNTPs or derivatives thereof.

The RNA polymerase can be any RNA polymerase able to incorporate S-2′-e-NTPs (preferably S-2′-F-NTPs or S-NTPs), or ara-NTPs or derivatives thereof and S-2′-e-3′-dNTPs (preferably S-3′-F-dNTPs or S-3′-dNTPs), ara-3′-dNTPs or derivatives thereof (Padilla, R.; Sousa R. Nucleic Acids Res. 1999, 27, 1561-1563; and Griffiths, A. D., et al., Nucleic Acids Res. 1987, 15, 4145-4162). Examples of suitable RNA polymerases are T7, T3, K11, SP6 and BA14 RNA polymerases (Hyone-Myong Eun, “Enzymology Primer for Recombinant DNA Technology” Academic Press, Inc., 1996, Chapter “RNA Polymerases”). Particularly advantageous for the TS method are the RNA polymerases having mutations as described in WO 99/02729, showing an enhanced ability for incorporating NTPs and/or 3′-NTPs. These mutant RNA polymerases are, for example, T7 RNA polymerase having at least one of the mutations F644Y, L665P, F667Y, F644Y/L665P, F644Y/F667Y, L665P/F667Y and F644Y/L665P/F667Y; a T3 RNA polymerase having at least one of the mutations F645Y, L666P, F668Y, F645Y/L666P, F645Y/F668Y, L6656/F668Y and F645Y/L666P/F668Y; a K11 RNA polymerase having at least one of the mutations L668P, F690Y, L688P/F690Y. Preferably, the RNA polymerase is a T7 RNA polymerase having the mutations F644Y and/or F667Y. A more complete description of suitable mutant RNA polymerases as well as the explanation of the terminology used can be found in WO 99/02729. A further useful RNA polymerase is the T7 RNA polymerase Y639F described in Padilla, R. and Sousa, R., Nucleic Acids Res., 1999, 27, 1561-1563.

Once the RNA transcript fragments are prepared according to the TS methodology known in the art (see reference above cited) and comprising the modified ribonucleotides according to the present invention, said RNA transcript fragments can be sequenced using MALDI-TOF-MS methodology.

The transcripts S-2′-e-RNA (preferably S-2′-F-RNA or S-RNA) which are produced by T7 RNA polymerase have only Rp-thiophosphodiester linkage. In order to maintain the Rp-S-linkage, they show the ability to resist to RNA cleavage, nucleaseS1, nucleasePI, RNaseT1 and RNaseA (Padilla, R.; Sousa R. Nucleic Acids Res. 1999, 27, 1561-1563; Dahm, S. C., et al., Biochemistry 1993, 32, 13040-13045; Loverix, S., et al., J. Chemistry & Biology 2000, 7, 651-658).

The present invention also relates to a method for the determination of SNPs using MALDI-TOF-MS and S-2′-e-NTPs (preferably S-2′-F-NTPs or S-NTPs) or ara-NTPs. The method for determining SNPs can be realized using the TS method and applying MALDI-TOF-MS as above described.

In particular, for determining polymorphism, at least two alleles (or one allele and a wild type) of the same gene or gene fragment have to be sequenced. Alternatively, one or more alleles are sequenced and compared to an already known sequenced allele (or to the wild type).

A general method and different approaches for determining SNPs using MALDI-TOF-MS is described in U.S. Pat. No. 5,965,363.

Preferably, the sequences or fragment transcripts (oligoribonucleotides) analyzed for polymorphism are subjected to accurate purification, in order to remove unwanted nucleic acid products from the spectrum. Further, the size of the oligoribonucleotides to be analyzed have to be within the range of the mass-spectrometry able to assure the necessary mass resolution and accuracy (see U.S. Pat. No. 5,965,363).

In a particular embodiment, the template DNA can be amplified, using specific primers, according to techniques known in the art. Then, a target sequence (that is a sequence that one intends to analyze and sequence comprising the presumed polymorphism) is distinguished. Accordingly, the target sequence (for example corresponding to an exon or shorter) will is extracted from the template preferably during the amplification step using the appropriate primers, or by cleaving off a portion of one or more flanking regions at the level of DNA template. Then, the transcription product is prepared and the masses of each of the reduced-length (amplified or not) target oligoribonucleotide(s) is determined using MALDI-TOF-MS. This method can be used to detect polymorphism in a single target nucleic acid by detecting variability in mass as compared to a wild type target nucleic acid or other alleles of said target nucleic acid.

The method can also be used to detect polymorphisms in a set of different target nucleic acids comprising (optionally also comprising amplifying each of said target nucleic acids) reducing the length and/or isolating a target oligonucleotide, using the TS method and determining the masses of the transcription products, comprising the incorporated ribonucleotides of the present invention, by MALDI-TOF-MS.

The invention further refers to a kit for sequencing a DNA template or a RNA transcription product by MALDI-TOF-MS, comprising:

i) a set of chain-elongating ribonucleosides triphosphates or alpha-phosphothioated for synthesizing a RNA transcription product, said chain-elongating ribonucleosides selected from the group consisting of S-2′-e-NTPs (preferably S-2′-F-NTPs or S-NTPs) and ara-NTPs;

ii) one or more chain-terminating ribonucleotide for terminating the synthesis of the RNA transcription product and generating sets of base-specific terminated complementary ribonucleotide transcription fragments; and

iii) a RNA polymerase.

The kit above disclosed can also optionally further comprise:

iv) a set of primers suitable for amplification of the template or target DNA; and

v) one or more matrix for MALDI-TOF-MS analysis. A matrix can be selected, for example, among those described in Zhu, Y. F., et al., Rapid Commun. Mass Spectrometry, 1996, 10, 383-388; Tang, W., et al., Anal. Chem., 1997, 69, 302-312.

The present invention also refers to a kit for the determination of SNPs using MALDI-TOF-MS comprising the same elements (i)-(iii) and the optional elements (iv)-(v) as disclosed for the kit for sequencing a DNA template or a RNA transcription product, and further optionally at least one restriction endonuclease capable of reducing the length of amplified target oligonucleotides (U.S. Pat. No. 596,363).

The products and methods according to the present invention using the MALDI-TOF-MS methodology will be particularly advantageous, for example, in the area of DNA re-sequencing and/or SNPs investigation, because the invention remedies the substantial phenomenon of signal intensity drop-off with increasing mass range.

The present invention will be further explained more in detail with reference to the following examples.

### EXAMPLES

Matrices and Setting of Mass Spectrometer

The matrices employed were purchased from Aldrich Chemical Co. (Milwaukee, Wis.). The preparation of matrix 3-hydroxypicolinic acid (3-HPA) followed the protocol, which mixed 180 μl of 0.5M 3-HPA in 50% acetonitrile and 20 !l of 0.5 M ammonium citrate dibasic in MilliQ water, was employed in experiments. The matrix 2,5-dihydroxybenzoic acid (2,5-DHBA) was prepared as a 0.5M solution in 10% acetonitrile. The preparation of 2,4,6-trihydroxyacetophenone (2,4,6-THAP)/2,3,4-trihydroxyacetophenone (2,3,4-THAP) matrix followed the protocol previously described (Zhu, Y. F., et al., Rapid Commun. Mass Spectrometry. 1996, 10, 383-388) and a molar ratio of 2:1:1 for 2,4,6-THAP:2,3,4-THAP:ammonium citrate dibasic was employed for all THAP preparations. Matrix 2,5-DHBA or THAP was employed to certify and investigate oligonucleotide fragmentation in MALDI process. Each 10, 20 and 30 mer oligonucleotide of 50 pmol per μl was mixed as the preparation of equimolar mixture. To an oligonucleotide sample was added a matrix solution, on a 2-mm diameter stainless steel probe tip. This solution was mixed well with pipetting on the probe tip and allowed to crystallize at room temperature before analysis by MALDI-TOF-MS.

Mass spectra were obtained on a Bruker Reflex III time-of-flight mass spectrometer, equipped with a 337 nm N₂ laser giving a 2 ns pulse width and operated in linear, positive-ion detection mode at 28.5 kV (IS/1) with a delayed extraction voltage of 20.8 kV (IS/2). The laser power setting employed for the samples was 25-30% of the full laser power. Sweet spots on the surfaces of the matrix and sample mixture crystallized were searched and shot to gain the best spectrum in all experiments. Clear crystals were favored over white muddy color crystals. Each spectrum consisted of the sum of 50 shots. MALDI-TOF-MS was performed as described according to the state of the art literature.

Preparation of Ribonucleotides and DNA, S-DNA, RNA, 2′-F-RNA, S-RNA Ladders

DNA, S-DNA, RNA and S-RNA (FIGS. 3A, 3E, 3B and 3F, respectively) ladders were synthesized and HPLC purified by GENSET OLIGOS (Kyoto, Japan and Paris, France). Sequence of the DNA were 10 mer: d(GATCTCAGCT) (SEQ ID NO:1);

| | |
|---|---|
| 20 mer: | |
| d(GATCTCAGCTCTAATGCGGT); | (SEQ ID NO:2) |
| 30 mer: | |
| d(GATCTCAGCTCTAATGCGGTTCGATAAATC). | (SEQ ID NO:3) |

Sequences of the RNA were the same to the DNA with the difference that T was U in the RNA and are defined as 10 mer: (GAUCUCAGCU) (SEQ ID NO:4), 20 mer: (GAUCUCAGCUCUAAUGCGGU) (SEQ ID NO:5), and 30 mer: (GAUCUCAGCUCUAAUGCGGUUCGAUAAAUC) (SEQ ID NO:6).

S-DNA and S-RNA were synthesized as phosphorothioate substituted the DNA and the RNA with amine at 3′ end for adding positively charged tag, respectively.

2′-fluoro-(C)ₙT (2′-F-RNA) (n=10, 20 or 30) were synthesized and purified from polyacrylamide gel by TriLink BioTechnologies (San Diego, Calif.) (SEQ ID NO: 7-9). In the following sequences SEQ ID NO: 7-9, the T in position 3′ was added with the only purpose of starting material on CPG beads (CPG, Inc., Lincoln Park, N.J. 07035) during the synthesis:

The dTTP is not 2′-modified and was not considered for the purpose of the present invention. Only the 2′-F-CTPs were counted for the numbering of the oligomers. Therefore, even is the oligomers synthesized are 11, 21 and 31 mer, they were referred to as 10 mer, 20 mer and 30 mer.

| | | |
|---|---|---|
| The 10mer: | 5′-2′-fluoro CCCCCCCCCCT-3′; | (SEQ ID NO:7) |
| The 20mer: | 5′-2′-fluoro CCCCCCCCCCCCCCCCCCCCT-3′; | (SEQ ID NO:8) |
| The 30mer: | 5′-2′-fluoro CCCCCCCCCCCCCCCCCCCCCCCCCCCCCCT-3′. | (SEQ ID NO:9) |

All oligonucleotides as stock solution were dissolved with TE buffer (10 mM Tris-Cl pH 8.0, 1 mM EDTA pH8.0). MilliQ water was employed at any other dilution step.

Analysis of DNA, RNA and 2′-F-RNA Ladders

FIGS. 1C,D and FIGS. 2C,D and 2A,B show that equimolar mixtures of the 10, 20 and 30 mer for each of the three different 2′ groups were prepared and analyzed using two different matrices in MALDI. In each Figure, signal intensity drop-off with increasing mass range was a positive trend. The behavior of the oligonucleotide ladders were repeated as the same trend with 3-HPA and THAP. In FIGS. 1D, 2D and 2B the spectra of 30 mer in DNA, RNA and 2′-F-RNA ladder using THAP show that the trend of stability to bass loss was followed as the order 2′-F-RNA>RNA>DNA. Previous studies mentioned the same trend that much more electronegative 2′ group provided greater stabilization of the oligonucleotide in MALDI analysis (Scalf, M. Ph.D. thesis, University of Wisconsin, Madison, Wis.; 2000). However, in the present example, the intensity of electronegativity at 2′ position exhibited no effect toward resistance to signal intensity drop-off.

Analysis of S-DNA Ladder

FIGS. 1A and 1B compared to FIGS. 1C and 1D show that the substitution of phosphorothioate in DNA facilitated excessive fragmentation when spectra of the 20 mer and the 30 mer was focussed. Signal intensity drop-off with increasing mass range as shown in the S-DNA ladder was much more dramatic than that in the DNA. Previous study also suggested that oligo S-DNA was not suitable for sequencing product in MALDI analysis (Schuette, J. M., et al., J. Pharm. Biomed. Anal. 1995, 13, 1195-1203).

Analysis of S-RNA Ladder

S-RNA ladder was analyzed in matrices 3-HPA and THAP. FIGS. 4A and 4B compared to FIGS. 2C and 2D show that the substitution of phosphorothioate in RNA gained a resistance toward decreasing signal intensity drop-off with increasing mass range using both matrices. The signal intensity of each peak in THAP was almost even.

Synthesis and Analysis of S-2′-F RNA Ladder

S-2′-fluoro-(C)ₙT (S-2′F-RNA) (FIG. 3G) as phosphorothioate substituted 2′-fluoro-(C)ₙT (SEQ ID NO: 7-9) was also prepared by TriLink BioTechnologies. The numbering of oligomers length was 10 mer, 20 mer and 30 mer, and the presence of T at the 3′ end was not considered for reason of numbering. Similarly, the crude oligomers represented in FIG. 6, are indicated as 1-10 mer, 1-20 mer and 1-30 mer, because the T at position 3′ end was not considered for reason of numbering.

The crude S-2′-F—(C)ₙTs (S-2′-F-RNAs) were synthesized and processed by ethanol precipitation to remove excess salt and exchanged to the sodium salt form (as purchased from TriLink Biotechnologies, San Diego, Calif.). Crude 10 mer, 20 mer, 30 mer S-2′-F-RNA were not subjected to purification. Therefore, the crude 10 mer, 20 mer, 30 mer S-2′-F-RNA contained 1-10 mer, 1-20 mer, 1-30 mer S-2′-F-RNA, respectively. These crude oligoribonucleotides were used for the experiment reported in FIG. 6.

Then, the same kind of crude oligoribonucleotides were purified by polyacrylamide gel and purified oligoribonucleotides of 10 mer, 20 mer and 30 mer (S-2′-F-RNA) were obtained. These purified oligoribonucleotides were used for the experiment reported in FIG. 5

In FIGS. 5A and 5B, the 10, 20, 30 mer ladders of S-2′-F-RNA show extremely sharp signal peaks, no base loss, and even signal intensity peaks using 3-HPA and THAP matrices. The signal to noise ratio was high enough to detect these signals.

FIGS. 6A and 6B show that the 10 mer and the 20 mer of the crude S-2′-F-RNA were analyzed in 3-HPA. In order to judge how the ladder spectra is resistant to signal intensity drop-off with increasing mass range, a comparison with the spectra of 2′-F-RNA ladder as a control was performed. In the spectra of 2′-F-RNA ladder as shown in FIG. 2A, the signal intensity of the 20 mer was assigned as 20 when the intensity of the 10 mer was 100. The intensity decreased five times with increasing the mass about two times. However, in the spectra of 20 mer S-2′-F-RNA as shown in FIG. 6B, the intensity of the 18 mer was assigned as 62 when the intensity of the 9 mer was 100. The intensity decreased by less than a factor of two with increasing the mass by a factor of about two. The number of the signal peaks from S-2′-F-RNA ladder was less effect for decreasing the signal intensity. This result demonstrates that S-2′-F-RNA has the robust trend of resistance to signal intensity drop-off with increasing mass range. FIG. 6C shows that the crude S-2′-F-RNA was analyzed in 3-HPA. Decreasing the signal intensity with mass range was also demonstrated. However, the resistance to decreasing the signal intensity was revealed in the condition of which the length of the product is longer, the number of the mole is smaller. It is expected that equimolar of S-2′-F-RNA ladder would be able to exhibit spectra of almost even signal intensity in MALDI analysis.

This data demonstrated that the S-2′-F-RNA as well as the S-RNA have an ability to resist to signal intensity drop-off.

Synthesis and Analysis of CH₃S-RNA

In order to verify the effect of the introduction of an alkyl-thio at the alpha position of a phosphoric group in RNA, the analysis of CH₃S-RNA was performed. Gut et al. presented a procedure for selective DNA alkylation and detection by mass spectrometry using 10 mer phosphorothioated DNA (Gut, I. G., et al., Rapid Commun. Mass Spectrometry 1997, 11, 43-50). The same procedure was employed for producing 10, 20, 30 mer of CH₃S-RNA with a positive charged tag. Accordingly, positively charged 3′ tagged RNA (CH₃S-Oligoribonucleotide-N⁺(CH₃)₃) were synthesized. All methylated phosphorothioates,

| | |
|---|---|
| 5′-(SCH₃)-GAUCUCAGCU-(CH₃)₃N⁺C₅H₁₀CONH—C₆H₁₂OPO₂3′; | (SEQ ID NO: 10) |
| 5′-(SCH₃)-GAUCUCAGCUCUAAUGCGGU-(CH₃)₃N⁺C₅H₁₀CONH—C₆H₁₂OPO₂3′; | (SEQ ID NO: 11) |
| 5′-(SCH₃)GAUCUCAGCUCUAAUGCGGUUCGAUAAAUC)-(CH₃)₃N⁺C₅H₁₀CONH—C₆H₁₂OPO₂3′; | (SEQ ID NO: 12) |

were synthesized according to Gut et al., using the CH₃I reaction. The reproducibility of the analysis was certified in the present MALDI technique using alpha-cyano-4-hydroxycinnamic acid methyl ester (CNME) as matrix.

In analysis of CH₃S-oligoribonucleotide-N⁺(CH₃)₃, equivolumes from each 10 mer, 20 mer, 30 mer of the CH₃S-RNA after the addition of the positive charged tag were mixed as the preparation of equimolar mixture.

FIGS. 7A,B show spectra of these oligo CH₃S-RNA. In the analysis of CH₃S-RNA as shown in FIGS. 7A,B, a number of shorter spectra instead of the expected intact parent ion peak were observed.

The control spectra of those were shown in FIGS. 4A,B as spectra of S-RNA.

This data shows that the introduction of an alkyl-thio group in the alpha-phosphoric group of ribonucleotides (FIG. 7) does not avoid signal intensity drop-off, while the introduction of only a thio- group (FIG. 4) shows a resistance to signal intensity drop-off. This is a further confirmation that the effect of signal drop-off resistance of S-NTPs was not predictable.

Analysis of Ara-RNA Ladder

Arabino-(C)ₙT ladders (n=10 mer, 20 mer and 30 mer) having the sequences of SEQ ID NO: 7-9, respectively, were synthesized and purified from polyacrylamide gel by TriLink BioTechnologies (San Diego, Calif.). Also in this case as for the synthesis of 2′-F-RNAs and S-2′-F-RNAs, the oligomer lengths were indicated as 10 mer, 20 mer and 30 mer, since the T at position 3′ end was not considered for reason of numbering. The dTTP, in fact, does not have the —OH at 2′-epimer position that the ara-NTPs have.

Arabinonucleic acids were mentioned more stable toward snake venom phosphodiesterase (SVDPE) hydrolysis than the ribonucleic acid derivatives; i.e., ara-RNA>RNA>2′-F-RNA (Noronha, A. M., et al., J. Biochemistry-2000, 39, 7050-7062). Since the present inventors focused on a trend of sugar-phosphate in oligonucleotide, the order in stability to SVDPE hydrolysis was investigated to suit stability to fragmentation in MALDI analysis. The behavior of the ara-RNA ladder in MALDI analysis was further certified in the matrices 3-HPA and THAP. FIG. 8A shows that extremely sharp signal peaks and the resistance to signal intensity drop-off with increasing mass range were observed using 3-HPA. The signal intensity of each peak was almost even. Ara-RNA ladder in matrix THAP was also resistant among the 20 mer and the 30 mer as shown in FIG. 8B. The ara-RNA ladder exhibited resistance to signal intensity drop-off with increasing mass range as shown in FIGS. 8A and 8B. It has been understood that phosphorothioate-substitution of RNA made backbone cleavage difficult. The effect was investigated to prove that the backbone cleavage at the sugar-phosphate would become one of the key roles toward decreasing signal intensity with increasing mass range in MALDI analysis.

FIG. 8, preferably FIG. 8A, shows that, contrary to the disclosure in the state of the art, ara-NTPs are able to resist signal intensity drop-off using MALDI-TOP-MS.

Sequencing Method

RNA transcript fragments of a specific template DNA fragment or sequence can be produced with TS methodology disclosed in the references as above indicated. The RNA transcript fragments can be treated with an amount of desalting acid solution and can be recovered substantially free from contaminants. The transcripts are then mixed with a matrix and crystallized. The RNA sequence is determined by MALDI-TOF-MS, according to the methodology known in the art. The sequence of the template DNA bases are then determined according to the transcript RNA bases.

All cited patents, publications and other materials referred to in this application are herein incorporated by reference.

The invention being thus described, it will be obvious that the same may be varied in many ways. Such variations are not to be regarded as a departure from the spirit and scope of the present invention, and all such modifications as would be obvious to one skilled in the art are intended to be included within the scope of the following claims.

1. A method for MALDI-TOF-MS analysis and/or sequencing of oligoribonucleotides comprising perfoming MALDI-TOF-MS analysis and/or sequencing using at least one kind of modified ribonucleotide being alpha-phosphorothioated and having an electronegative substituent at position 2′ of the ribose, and as nitrogenous base adenine, guanine, cytosine, uracil and/or their derivatives.

2. The method of claim 2, wherein said electronegative substituent is selected from the group consisting of F, Cl, NH₂, N₃ and OH.

3. The method according to claim 1, wherein said at least one ribonucleotide is selected from the group consisting of alpha-phosphorothioated-2′-fluoro-ATP, -CTP, -GTP, -UTP and derivatives thereof.

4. The method according to claim 1, wherein the MALDI-TOF-MS analysis and/or sequencing is performed in the presence of a suitable MALDI matrix.

5. The method of claim 4, wherein the matrix is 3-HPA, THAP, 2,5-DHBA or derivatives thereof.

6. A method for MALDI-TOF-MS analysis and/or sequencing of oligoribonucleotides comprising performing MALDI-TOF-MS analysis and/or sequencing using at least one kind of modified ribonucleotide being alpha-phosphorothioated and having an arabino group at 2′ position of the ribose, and as nitrogenous base adenine, guanine, cytosine, uracil and/or their derivatives.

7. The method according to claim 6, wherein the MALDI-TOF-MS analysis and/or sequencing is performed in the presence of a suitable MALDI matrix.

8. The method of claim 7, wherein the matrix is selected from the group consisting of 2,5-DHBA, 3-HPA, THAP, CNME and derivatives thereof.

9. A method for determining the DNA nucleotide sequence using MALDI-TOF-MS comprising:
a) providing chain-elongating ribonucleotides selected from alpha-phosphorothioated-2′-electroegative substituent NTPs, or arabino-2′-NTPs;
b) reacting said chain-elongating ribonucleotides with one or more kinds of 3′-dNTP derivatives, as chain terminating ribonucleotides, in the presence of RNA polymerase and a DNA template comprising a promoter sequence for the RNA polymerase to obtain an oligoribonucleotide transcription product; and
c) analyzing said oligoribonucleotide transcription product by MALDI-TOF-MS and determining the sequence of the transcription product and the DNA template.

10. The method of claim 9, wherein said electronegative substituent is selected from the group consisting of F, Cl, NH₂, N₃ and OH.

11. The method of claim 9, wherein said RNA polymerase is a mutant T7, T3, K11, SP6 and BA14 RNA polymerase having improved ability to incorporate ribonucleotides.

12. The method of claim 11, wherein said mutant RNA polymerase is T7 RNA polymerase having mutation F644Y and/or F667Y.

13. The method according to claim 9, wherein the MALDI-TOF-MS analysis and/or sequencing is performed in the presence of a suitable MALDI matrix.

14. The method of claim 13, wherein the matrix is selected from the group consisting of 2,5-DHBA, 3-HPA, THAP, CNME and derivatives thereof.

15. A method for determining polymorphism using MALDI-TOF-MS, comprising the steps a)-c) of claim 9, wherein the oligoribonucleotide transcription product of step c) comprises at least two alleles of the same gene, an allele and a wild type, or at least one allele to be compared to a known wild type.

16. A kit for analyzing and/or sequencing a DNA template or a RNA transcription product and/or for determining polymorphism by MALDI-TOF-MS, comprising:
i) a set of chain-elongating ribonucleotides selected from alpha-phosphorothioated-2′-electroegative substituent NTPs, or arabino-2′-NTPs for synthesizing a RNA transcription product;
ii) one or more kinds of 3′-dNTP derivatives, as chain-terminating ribonucleotides, for terminating the synthesis of the RNA transcription product and generating sets of base-specific terminated complementary ribonucleotide transcription fragments; and
iii) a RNA polymerase.

17. The kit of claim 16, wherein said electronegative substituent is selected from the group consisting of F, Cl, NH₂, N₃ and OH.

18. The kit of claim 16, wherein said RNA polymerase is a mutant T7, T3, K11, SP6 and BA14 RNA polymerase having improved ability to incorporate ribonucleotides.

19. The kit of claim 18, wherein said mutant RNA polymerase is T7 RNA polymerase having mutation F644Y and/or F667Y.

20. The kit of claim 16, further comprising one or more matrix for the MALDI-TOF-MS analysis.

21. The kit of claim 20, wherein the matrix is selected from the group consisting of 2,5-DHBA, 3-HPA, THAP, CNME and derivatives thereof.


SEQUENCE LISTING (12 sequences)

SEQ ID NO: 1 - DNA, 10 bp, Artificial Sequence
  Description of Artificial Sequence Synthetic DNA 10mer
gatctcagct

SEQ ID NO: 2 - DNA, 20 bp, Artificial Sequence
  Description of Artificial Sequence Synthetic DNA 20mer
gatctcagctctaatgcggt

SEQ ID NO: 3 - DNA, 30 bp, Artificial Sequence
  Description of Artificial Sequence Synthetic DNA 30mer
gatctcagctctaatgcggttcgataaatc

SEQ ID NO: 4 - RNA, 10 nt, Artificial Sequence
  Description of Artificial Sequence Synthetic RNA 10mer
gaucucagcu

SEQ ID NO: 5 - RNA, 20 nt, Artificial Sequence
  Description of Artificial Sequence Synthetic RNA 20mer
gaucucagcucuaaugcggu

SEQ ID NO: 6 - RNA, 30 nt, Artificial Sequence
  Description of Artificial Sequence Synthetic RNA 30mer
gaucucagcucuaaugcgguucgauaaauc

SEQ ID NO: 7 - RNA, 10 nt, Artificial Sequence
  Description of Artificial Sequence Synthetic RNA 10mer
cccccccccc

SEQ ID NO: 8 - RNA, 20 nt, Artificial Sequence
  Description of Artificial Sequence Synthetic RNA 20mer
cccccccccccccccccccc

SEQ ID NO: 9 - RNA, 30 nt, Artificial Sequence
  Description of Artificial Sequence Synthetic RNA 30mer
cccccccccccccccccccccccccccccc

SEQ ID NO: 10 - RNA, 10 nt, Artificial Sequence
  Description of Artificial Sequence Synthetic oligomer
gaucucagcu

SEQ ID NO: 11 - RNA, 20 nt, Artificial Sequence
  Description of Artificial Sequence Synthetic oligomer
gaucucagcucuaaugcggu

SEQ ID NO: 12 - RNA, 30 nt, Artificial Sequence; identical to SEQ ID NO: 6
  Description of Artificial Sequence Synthetic oligomer